SEQUENCE LISTING (5 sequences)

SEQ ID NO: 1 - DNA, 26 bp, Bos taurus
gtgctctaatcttttctggtaccagg

SEQ ID NO: 2 - DNA, 26 bp, Bos taurus
cctccccaaatcaattacattttctc

SEQ ID NO: 3 - DNA, 2869 bp, Bos taurus
ctcgaggatccttgttcgtgtccattttaaatatagaagtgtgttcatgtccatccccaaaaccctaactatctcttcctccagctttcctcccagcaaccataaattcattctctaaatctgtgagtctgttttgtaagtaagttcatttgtatcatttctttttagtttccacatataagagatgtcatacaatatttcctcttctctgtctgacttacttcactcagtatgacaatctctaggtcatccgtgttgctgcagatgacattatttcattctttttaatggccgagtaatatccagtgtgtgtgtgtgtgtgcgtgtgtttatatatacataccttctttatcctttcctctgtcaatggacattcagttactttcaggtcttggctgttgtaaacaatactgtaatgaacattggggtgcatgtatcctttcagtactagtttttctctgatatatagcccaagagtgagttagcagggtctataggtaacttttttaaggaacctccttacttttttccatagtgattgtgccaatttacattcccaccaacactgtaggaagatgaatggtcttcttgtattgggagcatggacaggaccattggtcatataagaataatactcacatagctttgcatgcaggcttgggtcatggctgactggtaaagaatctacctgccaaagcagagacacaggttcattccctgagtcgggaagatctcctggagaaggaaatcgtaaccccctgcagtgttcttgcctgggaaaccccatggacaaaggagcctggcaggctatagcccttgggtttgcaaaatcagacatgactgaataactagcagcaaagctttgcgtgcacagcagctcaacccacactcagtggtgggaatcattgtgattgttctaactggtgaggaggctacaggaaatctggtgaagctccagataatagccactgataggtactataattaaacatggaactttaagtatgttgggatctccaatgggcactaatgttttaaatttttttttttcttccaattttattttatttttaaactttacataattgtattagttttgccaaatatcaaaatgaatccgccacaggtatacatgtgttccccatcccgaaccctcctccctcctccctccccataccatccctctgggccgcccagtgctccagccccaagcatccagcatcatgcatcgaacctggactggcaactcgttcctacatgatatttcacatgtttcattgccattctcccaaatcttcccaccctctccctctcccacagagtccataagactgttctatacatgagtgtctcttttgctgtctcgtacaccgggttattgttaccatctttctaaatcccatatatatgcgttagtatactgtatttatgtttttccttctggcttacttcactctgtataataggctccagtttcatccacctcattagaactgattcaaatgtattctttttaatggctgagtaatactccattgtgtatatgtaccacagctttcttatccattcatctgctgatggacatctaggttgcttccatgtcctggctattataaacagtgctgcgatgaacattggggtacacgtgtctctttcccttctggtttcctcagtgtgtatgcccagcagtggggttgctggatcataaggcagttctatttccagttttttaaggaatctccacactgttctccatagtggctgtactagtttgcattcccaccaacagtgtaagagggttcccttttctccacaccctctccagcatttattatttgtagacttttggatcgcagccaatctgactggtgtgaaatggtacctcatagtggtttgatttgcatttctctgataatgagtgatgttgagcatcttttcatgtgtttgttagccatctgtatgtcttttttggagaaatgtctatttagttctttggcccattttttgattgggtcgtttatttttctggagttgagctgtaggagttgcttgtatatttttgagattagttgtttgtcggttgcttcatttgctattattttctcccattctgaaggctgtcttttcaccttgctaatagtttcctttgatgtgcagaagcttttaaggttaattaggtcccatttgtttatttttgcttttatttccaatattctgggaggtgggtctcccagaatgttttaaaatttaattgctcacccttcatttaacaaatattccacttgctatactctgggttcttgggatccttcatggagattccagcacctctgccctcctggagcttccttccttgaactccttagctgtgggattagattccgacaactctccctgtcttcagcccctctggcgtatggtctttgtcaaattctaatacgggccttctcagttggtctggctggccccatcctgatgagccttgtgagcctccagcccaggcctggccttcacttcagttggcagaacccagccctgggcaaaggtcggggggttcgttatgtgaggcaatgcgttgtgtgctctaatcttttctggtaccaggttgtgtgtgtgtgtgtgtgtgtgtgtgtgtgtgtgtgtgtgactgggagggaggaagagagagaaaatgtaattgatttggggaggatttggggaaggtttatataggaaagcagcaagaccaagaatctactgccaagcggtgaccaagaaacgttcaccatattcctcctccaaccccgcactgtttgccaactcttaaccaaattagcatagtgcggtctgcttccatacatgactgaatgaataaggaagtttagacgtc

SEQ ID NO: 4 - DNA, 540 bp, Bos taurus
ttagattccgacaactctccctgtcttcagcccctctggcgtatggtctttgtcaaattctaatacgtggccttctcagttggtctggctggccccatcctgatgagccttgtgagcctccagcccaggcctggccttcacttcagttggcagaacccagccctgggcaaaggtcggggggttcgttatgtgaggcaatgcgttgtgtgctctaatcttttctggtaccaggttgtgtgtgtgtgtgtgtgtgtgtgtgtgtgtgtgtgtgtgactgggagggaggaagagagagaaaatgtaattgatttggggaggatttggggaaggtttatataggaaagcagcaagaccaagaatctactgccaagcggtgaccaagaaacgttcaccatattcctcctccaaccccgcactgtttgccaactcttaaccaaattagcatagtgcggtctgcttccatacatgactgaatgaataaggaagtttagacgtccttgccataaagcctggaggaaccatacgaaaatccagcctctg

SEQ ID NO: 5 - DNA, 522 bp, Bos indicus
ttagattccgataactctccctgtcttcagcccctctggcgtatggtctttgtcaaattctaatacgtggccttctcagttggtctggctggctccatcctgatgagccttgtgagcctccagcccaggcctggccttcacttcagttggcagaacccagccctgggcaaaggtcggggggttcgttatgtgaggcaatgcgttgtgtgctctaatcttttctggtaccaggttgtgtgtgtgtgtgtgtgtgtgactgggagggaggaagagagagaaaatgtaattgatttggggaggatttggggaaggtttatataggaaagcagcaagaccaagaatctactgccaagcggtgaccaagaaacgttcaccatattcctcctccaaccccgcactgtttgccaactcttaaccaaattagcatagtgcggtctgcttccatacatgactgaatgaataaggaagtttaaacgtccttgccataaagcctggaggaaccatacgaaaatccagcctctg

(19) United States
(12) Patent Application Publication
(10) Pub. No.: US 2002/0142315 A1
(43) Pub. Date: Oct. 3, 2002

(54) DNA marker for cattle growth

(75) Inventors: Chad S. Hale, Corvallis, OR (US); William O. Herring, Dothan, AL (US); Gary S. Johnson, Ashland, MO (US); Duane H. Keisler, Columbia, MO (US); Dennis B. Lubahn, Columbia, MO (US); Matthew S. Lucy, Columbia, MO (US); H. Hisashi Shibuya, Kanagawa-ku (JP)

Correspondence Address: ROBERT E. HANSON, FULBRIGHT & JAWORSKI L.L.P., SUITE 2400, 600 CONGRESS AVENUE, SUITE 2400, AUSTIN, TX, 78701, US

(73) Assignee: The Curators of the University of Missouri

(21) Appl. No.: 09/910,428

(22) Filed: Jul. 19, 2001

Related U.S. Application Data
(60) Provisional application No. 60/219,180, filed on Jul. 19, 2000.

(51) Int. Cl. C12Q 1/68; A01K 67/027

(52) U.S. Cl. 435/6; 800/15

(57) ABSTRACT

The invention overcomes deficiencies in the prior art by identifying a locus associated with average weaning weight and carcass weight of cattle. The locus was found during studies carried out by the inventors using a polymorphic TG-repeat microsatellite located 90 base pairs upstream from a major transcription start site in the bovine growth hormone receptor gene. Use of this marker and other genetic markers in linkage disequilibrium with the locus allows implementation of selection and breeding schemes for improvement of cattle performance. Marker-assisted selection with the genetic markers will allow avoidance of potentially costly phenotypic testing associated with traditional breeding schemes.

### BACKGROUND OF THE INVENTION

This application claims the benefit of priority to U.S. Provisional Application Ser. No. 60/219,180 filed Jul. 19, 2000, which is specifically incorporated by reference in its entirety.

1. Field of the Invention

The present invention relates generally to the field of mammalian genetics. More particularly, it concerns genetic markers for the selection of cattle having a genetic predisposition for superior growth traits.

2. Description of Related Art

The growth hormone receptor genes in a variety of mammalian species contain three or more alternative first exons (Edens and Talamantes, 1998). The 5′ ends of transcripts from these genes consist of one of the alternative first exons spliced to a single second exon. Because the second exon contains the codon for the initiator methionine, the choice of the alternative first exon does not alter the structure of the product growth hormone receptor protein. Nonetheless, distinct promoters regulate transcription from each of the alternative first exons, thereby contributing to the complexity of growth hormone receptor expression, which varies according to tissue type and developmental stage (Schwartzbauer and Menon, 1998).

The promoter designated P1 regulates growth hormone receptor expression in the liver and is associated with exon 1A in sheep and cattle. The orthologous promoters are designated V1 in man and L1 in rodents (Schwartzbauer and Menon, 1998). In dairy cattle, the corresponding 5′ region of the growth hormone receptor gene has been associated milk-related traits (Aggrey et al., 1999).

A TG-repeat occurs in or near the liver-specific first exon of the growth hormone receptor gene in at least five mammalian species. The mouse repeat is only four TGs long and is situated 89 bp upstream from the transcription start site (Menon et al., 1995). The orthologous human sequence contains six consecutive TGs that are included in the 5-prime untranslated region rather than in the 5-prime flanking region because the start site for the human liver-specific first exon is shifted upstream (Pekhletsky et al., 1997). In an ovine sequence, the microsatellite consists of 18 consecutive TGs located 88 bp upstream from the transcription start site (O'Mahoney et al., 1994). In Bos taurus and Bos indicus cattle an orthologous TG-microsatellite is 90 bp upstream from exon 1A and is polymorphic (Heap et al., 1995; Lucy et al., 1998). An 11-TG-repeat allele of this locus commonly occurs in Bos indicus cattle. Alleles with 16 to 20 consecutive TGs were shown to be most common in taurine breeds (Lucy et al., 1998). However, Lucy et al. (1998) failed to identify any phenotypic traits associated with the TG repeat.

The aforementioned studies have helped to provide an understanding of bovine genetics. However, there is still a great need in the art for novel genetic tools for the creation of superior animals. In particular, there is a need for the identification of genetic markers which have been shown to be associated with important traits in cattle. The identification of such genetic markers would allow marker assisted selections to be made with those markers, thereby greatly increasing the productivity of breeding programs for the relevant trait.

### SUMMARY OF THE INVENTION

In one aspect, the invention provides a method of obtaining a head of beef cattle comprising a genetic predisposition for increased or decreased carcass or weaning weight, the method comprising the steps of: (a) assaying genetic material from at least a first head of beef cattle for a genetic polymorphism genetically linked to promoter P1 of exon 1A of the bovine growth hormone receptor gene, wherein the polymorphism is associated with increased or decreased carcass or weaning weight; and (b) selecting a head of beef cattle having the polymorphism. In particular embodiments of the invention, the genetic polymorphism may be further defined as genetically linked to exon 1A of the growth hormone receptor gene. The polymorphism also may be further defined as a polymorphism in a portion of the genome of the head of beef cattle corresponding to the nucleic acid sequence of SEQ ID NO: 3.

In another aspect of the invention, potentially any type of polymorphism can be used to detect the major effect locus identified by the inventors, including a restriction fragment length polymorphism, simple sequence length polymorphism, amplified fragment length polymorphism, single nucleotide polymorphism or isozyme. A preferred marker constitutes a simple sequence length polymorphism, and particularly a thymine-guanine dinucleotide repeat including the thymine-guanine dinucleotide repeat flanked by the nucleic acid sequences of SEQ ID NO. 1 and SEQ ID NO. 2. Selecting with this marker may comprise selecting a desired length of repeat, including a repeat of at least 12 copies, between about 16 and about 20 copies, greater than 20 copies, or less than 12 copies of the thymine-guanine dinucleotide repeat. Assaying may be carried, for example, with PCR. The amplified fragments can then be efficiently scored using gel electrophoresis to identify specific amplification products by size, or could be done another way.

The method may find use with any type of beef cattle, such as a Bos indicus or Bos taurus cattle. Traits that may be selected with the invention include increased carcass weight, decreased carcass weight, increased weaning weight and decreased weaning weight, as well as associated traits. Genetic material assayed may comprise, for example, genomic DNA. This can be obtained from cattle post-birth, or may be obtained from fetal animals, including from embryos in vitro. The selecting may comprise embryo transfer of the embryo, such that the first head of beef cattle is grown from the embryo.

In yet another aspect, the invention provides a method of breeding cattle to increase the probability of obtaining a progeny head of beef cattle having a genetic predisposition for increased or decreased carcass or weaning weight, the method comprising the steps of: (a) selecting a first parent head of beef cattle comprising a genetic polymorphism genetically linked to promoter P1 of exon 1A of the bovine growth hormone receptor gene, wherein the polymorphism is associated with increased or decreased carcass or weaning weight; and (b) breeding the first parent head of beef cattle with a second parent head of beef cattle to obtain at least a first progeny head of beef cattle comprising the polymorphism associated with increased or decreased carcass or weaning weight. The method may further comprise selecting the second parent head of beef cattle based on a genetic polymorphism genetically linked to promoter P1 of exon 1A of the bovine growth hormone receptor gene, wherein said polymorphism is associated with increased or decreased carcass or weaning weight.

In particular embodiments of the invention, the genetic polymorphism may be further defined as genetically linked to exon 1A of the growth hormone receptor gene. The polymorphism also may be further defined as a polymorphism in a portion of the genome of the head of beef cattle adjacent to the nucleic acid sequence of SEQ ID NO: 3.

In the method, one or both of the first parent head of beef cattle and the second parent head of beef cattle may be any beef cattle type, for example a Bos indicus or Bos taurus head of beef cattle. Traits that may be bred with the invention include increased carcass weight, decreased carcass weight, increased weaning weight and decreased weaning weight, as well as associated traits. In the cross, either the first or second parent may be the sire. The method may still further be defined as comprising crossing a progeny head of beef cattle with a third head of beef cattle to produce a second generation progeny head of beef cattle. The third head of beef cattle may be a parent of the progeny head of beef cattle or may be unrelated to the progeny head of beef cattle. In another embodiment of the invention, the aforementioned steps (a) and (b) are repeated from about 2 to about 10 times, wherein the first parent head of beef cattle is selected from a progeny head of beef cattle resulting from a previous repetition of step (a) and step (b) and wherein the second parent head of beef cattle is from a selected cattle breed into which one wishes to introduce said genetic predisposition for increased or decreased carcass or weaning weight. This technique will allow, for example, the introduction of the beneficial carcass or weaning weight characteristic into a genetic background otherwise lacking the trait but possessing other desirable traits.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Distribution of adjusted weaning weights of steers in each of the six half-sibling families and in all families (right). Squares represent long/long homozygotes (l/l). Circles represent short/long heterozygotes (s/l).

FIG. 2. Comparisons of adjusted mean birth weights, adjusted mean weaning weights, and estimated mean finishing weights for long/long homozygous steers (squares) and short/long heterozygous steers (circles).

FIG. 3. Comparison of taurine and indicine nucleotide sequences surrounding the polymorphic TG-repeat. Dashes show where the taurine and indicine nucleotides are identical. Stars indicate the absence of a nucleotide. The gray background marks the TG-repeat. Bold letters are from exon 1A. The taurine and indicine nucleotide sequences are given by SEQ ID NO: 4 and SEQ ID NO: 5, respectively.

### DESCRIPTION OF ILLUSTRATIVE EMBODIMENTS

The current invention overcomes deficiencies in the prior art by identifying a locus associated with the size of both juvenile and adult cattle. In particular, the inventors have identified a genetic locus linked to the upstream region of a major transcription start site in the bovine growth hormone receptor gene which is associated with phenotypic expression of beneficial growth characteristics including increased weaning and carcass weights. The locus was found during studies carried out by the inventors using a polymorphic TG-repeat microsatellite located 90 base pairs upstream from exon 1A of the bovine growth hormone receptor gene. The polymorphism is located within promoter P1 of growth hormone receptor gene exon 1A. The findings of the inventors represent an advance in that they allow implementation of novel techniques for the identification of cattle having a genetic predisposition for increased growth without the need for costly and potentially inaccurate phenotypic testing.

With the increasing costs associated with animal breeding and artificial insemination, each head of cattle produced represents a substantial investment of time and money. Traditional methods of breeding cattle have included standard breeding techniques in which sire progenies are studied. However, such techniques may lack accuracy due to environmental variance or scoring error. Further, complex gene action and interactions among genes can complicate breeding. Phenotypic selection often does not efficiently take into account such genetic variability. As such, there is a great need in the art for novel methods for the genetic evaluation of cattle performance.

The studies carried out by the inventors demonstrated that a polymorphic TG-repeat microsatellite located 90 base pairs upstream from a major transcription start site in the bovine growth hormone receptor gene is associated with increases in both weaning weight (17±4 kg; P<.01) and carcass weight (14±5 kg; P<.01). Further, approaching significance (P=.03) was the contrast for USDA marbling score (−.3±.2); whereas, no significant differences (P>.05) were detected for birth weight (.3±.6 kg), ribeye area (−.2±1 cm²), or carcass fat depth (−.01±07 cm). In the inventors' studies, genotyping was carried out on 64 Angus sires with respect to the above-mentioned poly-TG microsatellite, leading to the identification of six bulls that were heterozygous in that they had one short 11-TG allele and one of the longer alleles (16-20 TG repeats). The shorter allele with 11 consecutive TGs is common in Bos indicus cattle; whereas, longer 16- to 20-TG-repeat alleles predominate in Bos taurus breeds. The 125 steer progeny of these six heterozygous bulls were then grouped according to their genotypes. Only the longer 16- to 20-TG-repeat alleles were found in 73 steer progeny (long/long homozygotes); whereas, a short 11-TG allele was paired with one of the longer alleles in 52 progeny (short/long heterozygotes). Contrasts for the long/long homozygotes vs. the short/long heterozygotes were significant for weaning weight (17±4 kg; P <.001) and carcass weight (14±5 kg: P<.01).

The results of the inventors indicated that cattle having the 16-20 TG dinucleotide repeat marker genotype in the growth hormone receptor allele in Angus steers raised under commercial conditions exhibited increased growth by an average of approximately 17 kg at weaning and approximately 23 kg at slaughter relative to animals having the 11 TG marker genotype. These results indicate the potential for genetic marker-assisted selection to select for animals with increased growth potential. In particular, the use of markers linked to the major effect locus shown for growth rate here will find use in breeding or selecting of beef cattle produced for slaughter, e.g., for production of meat products. Thus, one embodiment of the invention comprises a breeding program directed at enhancement of growth characteristics in beef cattle breeds adapted for meat production, as opposed to cattle specifically suited or used for production of dairy products.

I. Marker-Assisted Selections in Accordance with the Invention

Marker assisted selection for animal breeding is important in that it allows selections to be made without the need for raising and phenotypic testing of progeny. In particular, it allows selection to occur among related individuals that do not exhibit the trait in question and that can be used in introgression strategies to select both for the trait to be introgressed and against undesirable background traits (Hillel et al., 1990). However, it is generally difficult to obtain genetic markers genetically linked to loci yielding highly heritable traits of large effect, particularly as many such traits may already be fixed with near optimal alleles in commercial lines. The invention overcomes this difficulty by providing such markers. Marker assisted selection also can be confounded by both recombination between the marker and the actual contributing locus and by mutation elsewhere in the genome (e.g., Keightley and Hill, 1992) whose effects are accommodated in classical selection, but are ignored in marker assisted selection. However, the tight linkage shown by the inventors relative to the trait of interest indicates that recombination is not a significant problem for selections in accordance herewith.

Here, the inventors have shown that a polymorphic TG-repeat microsatellite located 90 base pairs upstream from a major transcription start site in the bovine growth hormone receptor gene is associated with increased weaning and carcass weights. As such, this marker will find use in accordance with the invention in the selection of individuals having the desired growth trait. However, the invention is not limited to the use of this particular marker, as the identification of the marker association by the inventors will allow one of skill in the art to identify other genetic markers linked to the identified major effect locus. In particular, any genetic marker in linkage disequilibrium with the locus identified by the inventors may be used to select individual cattle having a genetic predisposition for increased growth. For example, other genetic markers or genes may be linked to the polymorphisms disclosed herein so that assays may involve identification of other genes or gene fragments, but which ultimately rely upon genetic characterization of animals for the same polymorphism. By “linked” or “genetically linked” it is meant that a marker locus and a second locus are sufficiently close on a chromosome that they will be inherited together in more than 50% of meioses, e.g., not randomly. Thus, the percent of recombination observed between the loci per generation (centimorgans (cM)), will be less than 50. In particular embodiments of the invention, genetically linked loci may be 45, 35, 25, 15, 10, 5, 4, 3, 2, or 1 or less cM apart on a chromosome. Preferably, the markers are less than 5 cM apart and most preferably about 0 cM apart.

Any assay which sorts and identifies animals based upon the allelic differences disclosed herein is included within the scope of this invention. One of skill in the art will recognize that once a polymorphism has been identified and a correlation to a particular trait proven, there are an essentially infinite number of ways to genotype animals for this polymorphism. The design of such alternative tests merely represents a variation of the techniques provided herein and is thus within the scope of this invention as fully described herein.

Once a marker system has been established, selections may be unambiguously made based on genotypes assayed at any time after a nucleic acid sample can be collected from an individual, such as an infant animal, or even earlier in the case of testing of embryos in vitro, or testing of fetal offspring. Any source of genetic material (including, for example, DNA and RNA) may be analyzed for scoring of genetic markers. In one embodiment of the invention, nucleic acids are screened which have been isolated from the blood or semen of the bovine analyzed. Generally, peripheral blood cells are used as the source, and the genetic material is DNA. A sufficient amount of cells are obtained to provide a sufficient amount of DNA for analysis, although only a minimal sample size will be needed where scoring is by amplification of nucleic acids. The DNA is isolated from the blood cells by standard nucleic acid isolation techniques known to those skilled in the art.

Any method of identifying the presence or absence of the marker may be used, including for example single-strand conformation polymorphism (SSCP) analysis, RFLP analysis, heteroduplex analysis, denaturing gradient gel electrophoresis, and temperature gradient electrophoresis, ligase chain reaction or even direct sequencing of the gene and examination for the certain recognition patterns. Techniques employing PCR detection are advantageous in that detection is more rapid, less labor intensive and requires smaller sample sizes.

In marker assisted breeding, eggs may be collected from selected females and in vitro fertilized using semen from selected males and implanted into other females for birth. Assays may be advantageously used with both male and female cattle. Using in vitro fertilization, genetic marker assays may be conducted on developing embryos at the 4-8 cell stage, for example, using PCR, and selections made accordingly. Embryos can thus be selected that are homozygous for the desired marker prior to embryo transfer.

Use of genetic marker-assisted selection may provide more efficient and accurate results than traditional methods. This also allows rapid introduction into or elimination from a particular genetic background of the specific trait or traits associated with the identified genetic marker. In the instant case, it was shown that a TG microsatellite marker was correlated with post-birth growth rates throughout development, including final carcass weight. As such, this marker and markers genetically linked to this marker will allow the efficient culling of low-weight-associated marker genotypes from breeding stock, as well as the introduction of higher-growth genotypes into genetic backgrounds lacking the trait, as desired.

Genetic markers can be used to obtain information about the genes which influence an important trait, thus facilitating breeding efforts. Factors considered in developing markers for a particular trait include: how many genes influence a trait, where the genes are located on the chromosomes (e.g., near which genetic markers), how much each locus affects the trait, whether the number of copies has an effect (gene dosage), pleiotropy, environmental sensitivity and epistatis.

The chromosomal location of a gene is determined by identifying nearby genetic markers which are usually cotransmitted with the gene from parent to progeny. This principle applies both to genes with large effects on phenotype (simply inherited traits) and genes with small effects on phenotype. As such, by identifying a single marker linked to a particular trait, this facilitates the development of additional markers linked to that trait. These markers also will have predicative power relative to the trait to the extent that they also are linked to the contributing locus for the trait. Such markers may even be more closely linked to the target locus and thus have greater predictive potential for the trait of interest.

A genetic map represents the relative order of genetic markers, and their relative distances from one another, along each chromosome of an organism. During sexual reproduction in higher organisms, the two copies of each chromosome pair, aligning themselves closely with one another. Genetic markers which lie close to one another on the chromosome are seldom recombined, and thus are usually found together in the same progeny individuals. Markers which lie close together show a small percent recombination, and are said to be linked. Markers linked to loci having phenotypic effects are particularly important in that they may be used for selection of individuals having the desired trait.

An important application for the genetic markers of the invention comprises animal breeding for beneficial growth characteristics. Such growth characteristics may comprise increased or decreased size, or other traits associated with the expression of these traits. Genetic markers represent genetic variation, permitting one to estimate relatedness between different genotypes, and consequently to predict which matings might produce new and superior gene combinations, in particular, having one or more selected genetic marker genotypes. For example, by having markers for loci of interest conferring a desired trait, one can readily detect recombination between these genes, and perform accurate selection for genetically superior individuals, from among the masses of candidates including many false positives resulting from environment.

Once linked markers are obtained, one can assay the marker genotype and predict with high likelihood whether the gene is present or absent, even before the trait can actually be seen. Further, many traits may be more accurately selected for by using genetic DNA markers than by relying solely on appearance, which may be due either to genotype or to environment.

Most natural populations of animals are genetically quite different from the classical linkage mapping populations. While linkage mapping populations are commonly derived from two-generation crosses between two parents, many natural populations are derived from multi-generation matings between an assortment of different parents, resulting in a massive reshuffling of genes. Individuals in such populations carry a complex mosaic of genes, derived from a number of different founders of the population. Gene frequencies in the population as a whole may be modified by a natural or artificial selection, or by genetic drift (e.g., chance) in small populations. Given such a complex population with superior average expression of a trait, a breeder might wish to (1) maintain or improve the expression of the trait of interest, while maintaining desirable levels of other traits; and (2) maintain sufficient genetic diversity that rare desirable alleles influencing the trait(s) of interest are not lost before their frequency can be increased by selection. Genetic markers may find particular utility in accomplishing this second objective; for example, one might select a fraction of the population based on favorable phenotype (perhaps for several traits-one might readily employ index selection), then apply genetic markers to this fraction and keep a subset which represent much of the allelic diversity within the population. Strategies for extracting a maximum of desirable phenotypic variation from complex populations remain an important area of breeding strategy. An integrated approach, merging classical phenotypic selection with a genetic marker-based analysis, may aid in extracting valuable genes from heterogeneous populations.

The techniques of the present invention may potentially be used with any bovine, including Bos taurus, Bos indicus cattle. In particular embodiments of the invention, the techniques described herein are specifically applied for selection of beef cattle, as the genetic markers described herein and linked to growth rate will find utility in maximizing production of animal products, such as meat. As used herein, the term “beef cattle” refers to any cattle which is grown or bred for production of meat or other non-dairy animal products. Therefore, a “head of beef cattle” refers to at least a first bovine animal grown or bred for production of meat or other non-dairy animal products. Examples of breeds of beef cattle that may be used with the current invention include, but are not necessarily limited to Africander, Albères, Alentejana, American, American White Park, Amerifax, Amrit Mahal, Anatolian Black, Andalusian Black, Andalusian Grey, Angeln, Angus, Ankole, Ankole-Watusi, Argentine Criollo, Asturian Mountain, Asturian Valley, Australian Braford, Australian Lowline, Ba-Bg, Bachaur, Baladi, Barka, Barzona, Bazadais, Beefalo, Beefrnaker, Beefinaster, Belarus, Red, Belgian Blue, Belgian Red, Belmont Adaptaur, Belmont Red, Belted Galloway, Bengali, Berrendas, Bh-Bz, Bhagnari, Blanco Orejinegro, Blonde d'Aquitaine, Bonsmara, Boran, Braford, Brahman, Brahmousin, Brangus, Braunvieh, British White, Busa, Cachena, Canary Island, Canchim, Carinthian Blond, Caucasian, Channi, Charbray, Charolais, Chianina, Cholistani, Corriente, Costeño con Cuemos, Dajal, Damietta, Dangi, Deoni, Devon, Dexter, Dhanni, Dølafe, Droughtmaster, Dulong, East Anatolian Red, Enderby Island, English Longhorn, Evolène, Fighting Bull, Florida Cracker/Pineywoods, Galician Blond, Galloway, Gaolao, Gascon, Gelbray, Gelbvieh, German Angus, German Red Pied, Gir, Glan, Greek Shorthorn, Guzerat, Hallikar, Hariana, Hays Converter, Hereford, Herens, Highland, Hinterwald, Holando-Argentino, Horro, Hungarian Grey, Indo-Brazilian, Irish Moiled, Israeli Red, Jamaica Black, Jamaica Red, Jaulan, Kangayam, Kankrej, Kazakh, Kenwariya, Kerry, Kherigarh, Khillari, Krishna Valley, Kurdi, Kuri, Limousin, Lincoln Red, Lohani, Luing, Maine Anjou, Malvi, Mandalong, Marchigiana, Masai, Mashona, Mewati, Mirandesa, Mongolian, Morucha, Murboden, Murray Grey, Nagori, N'dama, Nelore, Nguni, Nimari, Ongole, Orma Boran, Oropa, Parthenais, Philippine Native, Polish Red, Polled Hereford, Ponwar, Piedmontese, Pinzgauer, Qinchuan, Räitien Gray, Rath, Rathi, Red Angus, Red Brangus, Red Poll, Retinta, Rojhan, Romagnola, Romosinuano, RX3, Sa-Sg, Sahiwal, Salers, Salorn, Sanhe, Santa Cruz, Santa Gertrudis, San Martinero, Sarabi, Senepol, Sh-Sz, Sharabi, Shorthorn, Simbrah, Sirnmental, Siri, Slovenian Cika, South Devon, Sussex, Swedish Red Polled, Tarentaise, Telemark, Texas Longhorn, Texon, Tharparkar, Tswana, Tuli, Ukrainian Beef, Ukrainian Grey, Ukrainian Whitehead, Umblachery, Ural Black Pied, Vestland Red Polled, Vosges, Wagyu, Welsh Black, White Cáceres, White Park, Xinjiang Brown and Yanbian cattle breeds, as well as animals bred therefrom and related thereto.

II. Preferred Genetic Markers for Use with the Invention

The association reported herein was identified using a polymorphic TG-repeat microsatellite located 90 base pairs upstream from a major transcription start site in the bovine growth hormone receptor gene. In particular, the TG repeat is located within the P1 promoter of exon 1A of the somatotropin receptor gene. The nucleic acid sequence comprising this promoter region and exon 1A of the receptor gene is given by Genbank Accession No. U15731 (SEQ ID NO: 3, Heap et al., 1995).

The TG repeat marker constitutes a preferred genetic marker for use with the invention. Also preferred will be other genetic polymorphisms from within the genomic region corresponding to the nucleic acid sequence of SEQ ID NO: 3. The association shown here between the 11-TG allele and decreased growth in Angus steers may be directly attributable to the relatively short length of this TG repeat. Soller and colleagues reviewed earlier reports of microsatellites that influence transcription rates and concluded that microsatellite length polymorphisms are an important source of quantitative trait variation (Kashi et al., 1997; King et al., 1997). More recent studies also support this conclusion. For instance, incremental decreases in transcription rates were produced by step-wise increases in the repeat number from 16 to 20 for a CA-microsatellite located near an enhancer element in intron 1 of the human epidermal growth factor receptor gene (Gebhardt et al., 1999). In addition, step-wise increases in the repeat number from zero to 21 for a CA-microsatellite located in the promoter of the human matrix metalloproteinase 9 gene produced incremental increases in transcription rates (Shimajiri et al., 1999).

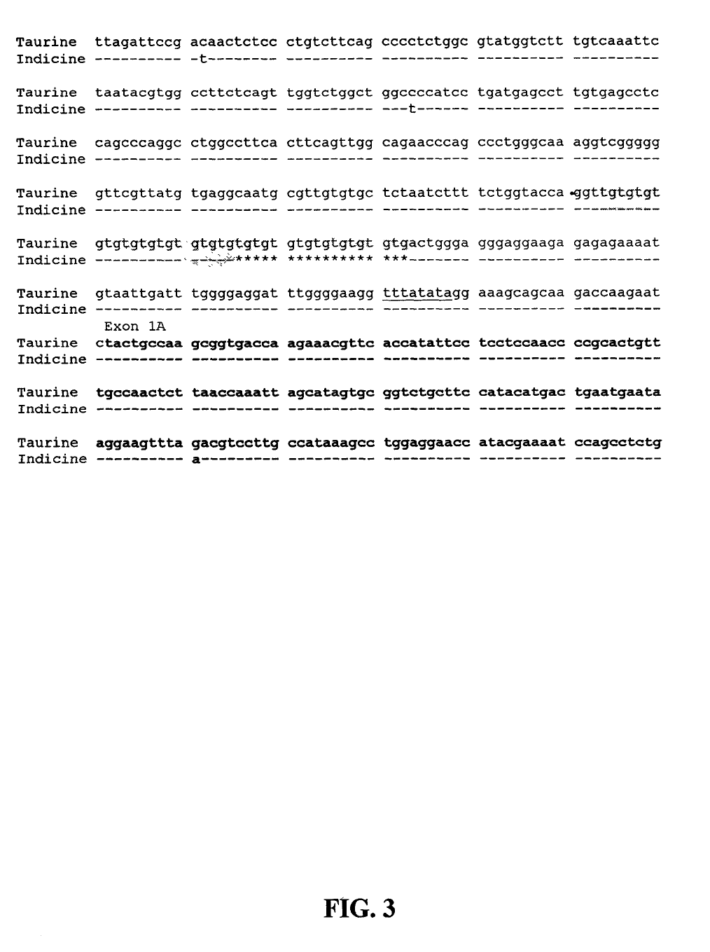

In a similar manner, the length of the TG-microsatellite in the 5-prime flanking region of bovine exon 1A may influence rates of growth hormone receptor transcript production because of its seemingly critical location. Although at least three distinct promoters regulate transcription of the bovine growth hormone receptor gene (Jiang et al., 1999), it appears that the promoter associated with exon 1A is important for regulating postnatal growth (Liu et al., 1999). In this promoter the TG-microsatellite is just 69 bp upstream from the TATA box (FIG. 3) and is flanked by nuclear protein binding sites, demonstrated by DNase 1 footprinting analysis and electromobility shift assays. On the other hand, an experiment by O'Mahoney et al. (1994) casts doubt on the notion that the TG-microsatellite influences growth hormone receptor transcription. These investigators studied the promoter for ovine exon 1A, which shares 94% sequence identity with the bovine promoter. They showed that deletion of a 104 bp segment, including the entire TG-repeat, from the ovine promoter had no significant effect on transcription rates in a human hepatoma cell line.

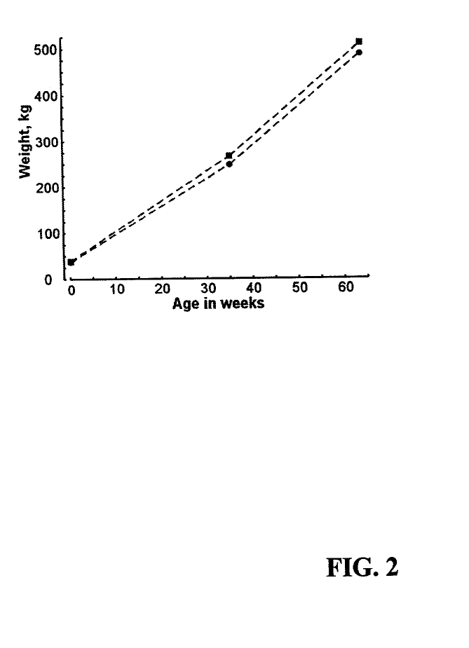

Alternatively, the 11-TG-repeat allele may be in linkage disequilibrium with proximal alleles that are directly responsible for decreased growth. In fact, the 11-TG-repeat allele is part of an indicus growth-hormone-receptor haplotype that also includes two single-base substitutions upstream from the TG-repeat and a downstream single-base substitution in exon 1A (FIG. 2). In addition, 0.35 kb upstream from the poly-TG microsatellite, the taurine haplotype has a 1.2 kb LINE retroposon which is absent from the indicine haplotype (Lucy et al., 1998).

Contamination of the Bos taurus genome with Bos indicus nucleotide sequences is likely to be widespread as crossbreeding of the two species has been occurring for the many thousands of years since both species were domesticated and could be transported around geographic barriers (Bradley et al., 1998). Lagziel et al. (1998) associated the indicine growth hormone haplotype with increased milk protein concentrations in taurine dairy cattle. They predicted that indicine haplotypes at other candidate loci would affect economically important traits and could be used to improve the taurine breeds. From the present study, the indicine growth hormone receptor haplotype appears to have a disadvantageous effect on growth so that the taurine breeds could be improved by marker-assisted selection away from the indicine haplotype.

The impact of the indicine allele on taurine beef yield cannot be accurately estimated until there is a better indication of the overall frequency of the 11-TG allele in taurine beef cattle. In addition, there is currently no information on magnitude of the growth effect of the indicine allele in other taurine breeds and in cattle kept in conditions that differ from those described here. Another consideration is that the indicine allele may have either a positive or a negative effect on additional quantitative traits. The surprisingly high indicine-allele frequency (.05) in the 64 Angus sires could have resulted from positive selection based on carcass quality or reproductive performance. Although not as significant as the growth effects, the mean USDA marbling score was higher for the heterozygous carcasses (Table 2). On the other hand, the low ratio of heterozygotes to homozygotes among the half siblings (52/73=.71) opens the possibility that the indicine allele is associated not only with decreased growth but also with decreased reproductive success and/or offspring viability.

III. Genetic Markers

Use of genetic markers forms an important part of the current invention. As described herein above, a preferred genetic marker that may be used with the invention comprises the polymorphic TG-repeat microsatellite located 90 base pairs upstream of exon 1A of the somatotropin receptor gene. However, other genetic markers may be used to detect this polymorphism in accordance with the invention.

Genetic markers are simply detected differences in the genetic information carried by two or more individuals. Genetic mapping of a locus with genetic markers typically requires two fundamental components: detectably polymorphic alleles and recombination or segregation of those alleles. In eukaryotes, the recombination measured is virtually always meiotic, and therefore, the two inherent requirements of animal gene mapping are polymorphic genetic markers and one or more families in which those alleles are segregating.

Markers are preferably inherited in codominant fashion so that the presence of both alleles at a diploid locus is readily detectable, and they are free of environmental variation, i.e., their heritability is 1. A marker genotype typically comprises two marker alleles at each locus. The marker allelic composition of each locus can be either homozygous or heterozygous. Homozygosity is a condition where both alleles at a locus are characterized by the same nucleotide sequence. Heterozygosity refers to different conditions of the gene at a locus. Exemplary genetic markers for use with the invention include, but are not limited to, restriction fragment length polymorphisms (RFLPs), simple sequence length polymorphisms (SSLPs), amplified fragment length polymorphisms (AFLPs), single nucleotide polymorphisms (SNPs), and isozymes.

Restriction fragment length polymorphisms (RFLPs) are genetic differences detectable by DNA fragment lengths, typically revealed by agarose gel electrophoresis, after restriction endonuclease digestion of DNA. There are large numbers of restriction endonucleases available, characterized by their nucleotide cleavage sites and their source, e.g., EcoRI. RFLPs result from both single-bp polymorphisms within restriction site sequences and measurable insertions or deletions within a given restriction fragment RFLP are easy and relatively inexpensive to generate (require a cloned DNA, but no sequence) and are co-dominant. RFLPs have the disadvantage of being labor-intensive in the typing stage, although this can be alleviated to some extent by multiplexing many of the tasks and reutilization of blots. Most RFLP are biallelic and of lesser polymorphic content than microsatellites. For these reasons, the use of RFLP in animal gene maps has waned.

Microsatellites (also called simple sequence length polymorphisms (SSLPs)) are tandem repeats of one to six bp, which are interspersed throughout the DNA of animal genomes (Litt and Luty, 1989; Tautz, 1989; Weber and May, 1989). Microsatellites have the advantage of being multi-allelic, highly polymorphic, co-dominant, and assayable by PCR. They have become the marker of choice of animal gene mapping projects. Each microsatellite region must initially be cloned and the surrounding sequence determined, but once this is done, these markers can usually be employed in many different resource populations, due to their high level of polymorphism. The sequence of the polymorphism itself, usually a single bp change, can be assayed in several ways. For example, it can be detected by electrophoretic techniques including a single strand conformational polymorphism (Orita et al., 1989), denaturing gradient gel electrophoresis (Myers et al., 1985), or cleavage fragment length polymorphisms (Life Technologies, Inc., Gathersberg, Md. 20877), but the widespread availability of DNA sequencing machines often makes it easier to just sequence amplified products directly. Once the polymorphic sequence difference is known, rapid assays can be designed for progeny testing, typically involving some version of PCR amplification of specific alleles (PASA, Sommer, et al., 1992), or PCR amplification of multiple specific alleles (PAMSA, Dutton and Sommer, 1991).

RAPD markers constitute another marker type that can be used for genetic mapping. RAPD markers derive from the fact that short (e.g., 10 mer) oligonucleotide primers in PCR reactions with lowered annealing criteria will generally amplify a spectrum of fragments from almost any template DNA. One or more of these fragments is often polymorphic (usually, but not always, due to a single base change in the primer binding site) and this polymorphism can be genetically mapped. Because large panels of RAPD primers can be purchased at reasonable cost from commercial suppliers, once again the upfront investment for RAPD mapping is low.

RAPD markers are dominant, which can be a limitation. RAPD markers are typically fairly evenly distributed throughout a genome and RAPD-generated polymorphic bands can be readily cloned for further analysis. Once the fragment is cloned, the source of the polymorphism can be examined by sequence analysis of the corresponding region of the parental genomes, which basically converts the RAPD to an STS (Okimoto and Dodgson, 1996). However, a major problem with RAPD patterns is their dependence on the exact PCR conditions employed, which can lead to reproducibility problems. This reduced reproducibility is probably due to the fact that the outcome of the amplification is extremely sensitive to the competition of inexact primer binding sites in the template for primers and polymerase in the critical early cycles. In this regard, RAPD patterns should be generated using at least two DNA template concentrations and a portion of each reaction should be stored for later cloning the fragment of interest, if necessary.

IV. Nucleic Acid Detection

Techniques for nucleic acid detection may find use in certain embodiments of the invention. For example, such techniques may find use in scoring individuals for marker genotypes or in the development of novel markers linked to the major effect locus identified herein.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, nucleotide sequences may be used in accordance with the invention for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, lower stringency conditions may be preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. For example, such techniques may be used for scoring of RFLP marker genotype. A wide variety of appropriate indicator means are known in the art, including fluorescent, radioactive, enzymatic or other ligands, such as avidin/biotin, which are capable of being detected. In certain embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific hybridization with complementary nucleic acid containing samples.

In general, it is envisioned that probes or primers will be useful as reagents in solution hybridization, as in PCR™, for detection of nucleic acids, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Nucleic acids used as a template for amplification may be isolated from cells, tissues or other samples according to standard methodologies (Sambrook et al., 1989). Such embodiments may find particular use with the invention, for example, in the detection of repeat length polymorphisms, such as microsatellite markers. In certain embodiments of the invention, amplification analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994). Typically, scoring of repeat length polymorphisms will be done based on the size of the resulting amplification product.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to obtain cDNA, which in turn may be scored for polymorphisms. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, also may be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, also may be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site also may be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., 1990; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids also may be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Other Assays

Other methods for genetic screening may be used within the scope of the present invention, for example, to detect polymorphisms in genomic DNA, CDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

5. Kits All the essential materials and/or reagents required for screening cattle for genetic marker genotype in accordance with the invention may be assembled together in a kit. This generally will comprise a probe or primers designed to hybridize specifically to individual nucleic acids of interest in the practice of the present invention, for example, primer sequences such as those of SEQ ID. NO. 1 and SEQ ID NO. 2 or of another nucleic acid sequence of SEQ ID NO: 3. Also included may be enzymes suitable for amplifying nucleic acids, including various polymerases (reverse transcriptase, Taq, etc.), deoxynucleotides and buffers to provide the necessary reaction mixture for amplification. Such kits also may include enzymes and other reagents suitable for detection of specific nucleic acids or amplification products. Such kits generally will comprise, in suitable means, distinct containers for each individual reagent or enzyme as well as for each probe or primer pair.

V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Correlation of Genetic Markers With Cattle Growth

Most of the animals studied were steer offspring produced over a 3-yr period from a herd of 6,000 commercial Angus cows used in an Angus sire-progeny-testing program. The Angus dams were bred randomly to young sires with some sires cross-classified across years. Resulting progeny were born in the spring on one of three Missouri ranches near Iberia, Stockton or Huntsville. After a short backgrounding phase at the ranch of origin, steer calves were managed on a silage-based ration to gain approximately 1 kg/d. Thereafter, they were transported to a commercial feedyard to be fed until slaughter. Calves were weighed at birth and weaning. At slaughter, the carcass data collected included carcass weight, ribeye area, carcass fat depth, and USDA marbling score.

Phenotypic data and blood samples for DNA isolation were gathered from over 2,000 steers sired by 64 Angus bulls from the progeny-testing program. Semen was obtained from all 64 sires. Washed sperm cells and leukocytes were lysed with proteinase K and dithiothreitol. Routine phenol-chloroform extraction and ethanol precipitation were used to isolate the DNA from these lysates. The DNA samples were suspended in tris/EDTA buffer and stored at −20° C. until they were genotyped with respect to the growth hormone receptor poly-TG microsatellite by a modification of the method of Weber and May (1989), as described by Lucy et al. (1998) and as follows.

The forward primer (5′GTGCTCTAATCTTTTCTGGTACCAGG-3′; SEQ ID NO: 1) was 32^{P}-labeled with T4 polynucleotide kinase. The 10 μL PCR amplification mixture contained 10 ng of genomic DNA, .5 U of Taq Polymerase, forward and reverse primers (reverse primer: 5′-CCTCCCCAAATCAATTACATTTTCTC-3′; SEQ ID NO: 2) (each 12.5 M), MgCl₂ (1.5 M). The thermal cycler program was 94° C. for 20 s, 62° C. for 30 s, and 72° C. 30 s for one cycle followed by 27 cycles of 94° C. for 20 s, 62° C. for 20 s, and 72° C. for 30 s. The PCR products were frationated by electrophoresis in a 4.0% denaturing polyacrylamide gel. Bands were visualized by routine autoradiography.

Based on the genotype results, the Angus steers were classified into two groups: the “long/long homozygotes” that contained only the longer 16- to 20-TG alleles and the “short/long heterozygotes” that contained one 11-TG allele and one 16- to 20-TG allele.

The computer program, MTDFREML (Boldman et al., 1993), was used to estimate long/long homozygote-short/long heterozygote contrasts for birth weight, weaning weight, carcass weight, ribeye area, carcass fat depth and marbling score. The data were analyzed using single-trait animal models that included fixed effects of contemporary group, age-of-dam, genotype within sire and a random animal effect. For birth weight, contemporary group was defined as ranch of birth and birth year. For weaning weight, contemporary group was defined as birth contemporary group and rearing pasture. For carcass traits, contemporary group was defined as weaning weight contemporary group, feedlot pen and slaughter date. Linear covariates of weaning age and slaughter age were included in the respective weaning weight and carcass trait models. First, contrasts (homozygote-heterozygote) were performed for genotype within sire, and then because the effect of genotype was homogeneous across sires, simply for genotype. Adjusted means were calculated using the model solutions.

### EXAMPLE 2

### Results

In a previous study, it was found that 9 of 9 DNA samples from Bos indicus cattle (Brahman and Nelore) contained only the 11-TG-repeat growth hormone receptor allele. In this study, fifty-eight of the 64 Angus sires analyzed contained only the longer 16- to 20-TG-repeat alleles. The remaining six sires were heterozygotes containing one 11-TG-repeat allele and one longer 16- to 20-TG-repeat allele.

Half-sibling steer offspring were available from each of the six heterozygous Angus bull sires. However, the number of steer offspring in these six half sibling families varied from 3 to 58. A total of 125 steer offspring were available for study; 73 were long/long homozygotes and 52 were short/long heterozygotes.

Unadjusted means (and standard deviations) for phenotypic data from the 73 long/long homozygous steers and the 52 short/long heterozygous steers are provided in Table 1. As can be seen, the mean weaning weights and the mean carcass weights of the long/long homozygous steers were greater than those of the short/long heterozygotes by 25 and 12 kg, respectively.

TABLE 1
Unadjusted weights, carcass characteristics, and management data
for 125 half-sibling steers from six sires

| | Long/long | | Short/long | |
|---|---|---|---|---|
| | homozygotes (n = 73) | | heterozygotes (n = 52) | |
| | Mean | S.D. | Mean | S.D. |
| Birth weight, kg | 38.5 | 5.5 | 38.5 | 4.4 |
| Weaning weight, kg | 271 | 41 | 255 | 37 |
| Carcass weight, kg | 321 | 34 | 309 | 33 |
| Carcass fat depth, cm | 1.18 | .41 | 1.26 | .43 |
| Ribeye area, cm² | 75.0 | 7.5 | 76.2 | 7.5 |
| Marbling score^{a} | 5.5 | .9 | 5.8 | 1.0 |
| Weaning age, weeks | 35.3 | 5.4 | 35.4 | 5.0 |
| Slaughter age, weeks | 64.1 | 2.6 | 63.3 | 3.0 |

^{a}4.0 = Slight⁰; 5.0 = Small⁰; etc.

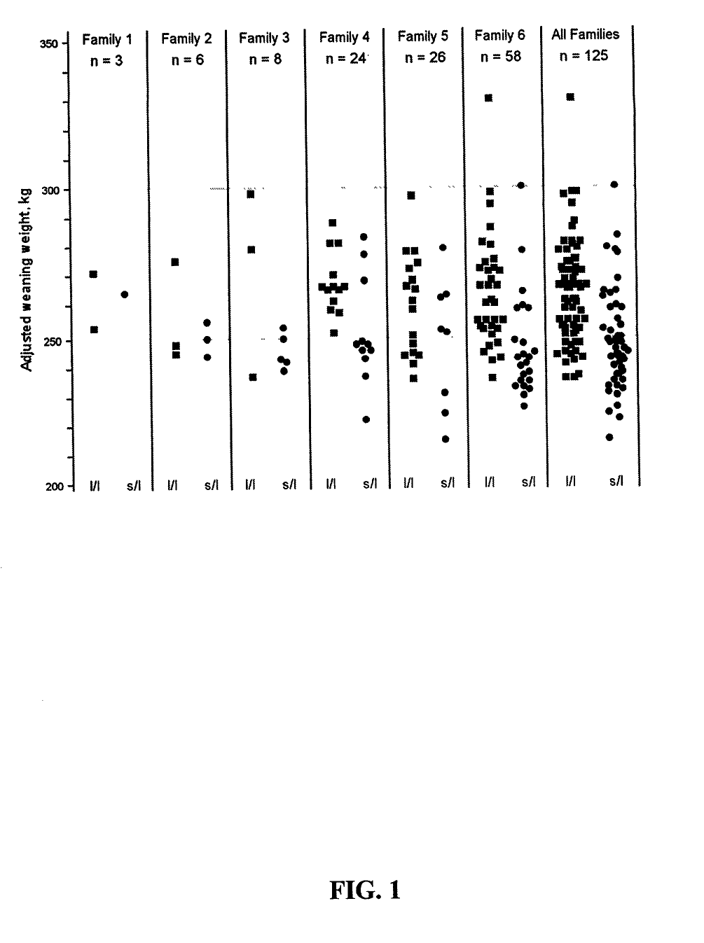

Adjusted means and contrasts are shown in Table 2. Contrasts for weaning weight (P<.001) and carcass weight (P<.01) were significant while the contrast for marbling score approached significance (P=.03). Contrasts in birth weight, carcass fat depth, and ribeye area were not significant (P>.05). In FIG. 1, the distributions of the adjusted weaning weights for individual long/long homozygotes are compared to those of the short/long heterozygotes.

TABLE 2
Trait impact of the long/long genotype (l/l) vs short/long
genotype (s/I) in 125 steers from six sires

| | Weights, kg | | | Ribeye | Carcass fat | |
|---|---|---|---|---|---|---|
| | Birth | Weaning | Carcass | Area, cm² | depth, cm | Marbling score^{a} |
| l/l mean (n = 73) | 38.8 | 265 | 316 | 76.1 | 1.22 | 5.6 |
| s/l mean (n = 52) | 38.5 | 248 | 302 | 76.3 | 1.23 | 5.9 |
| Contrast ± SE | 3 ± .6 | 17** ± 4 | 14** ± 5 | −.2 ± 1.0 | −.01 ± .07 | −.3* ± .2 |

^{a}4.0 = Slight⁰; 5.0 = Small⁰; etc.
^{***P < .001}
^{**P < .01}
^{*P < .05}

Although the steers were not weighed before slaughter, finishing weights were estimated by assuming 62% dressing percentage as suggested by Boggs and Merkel (1984). FIG. 2 compares the homozygotes and heterozygotes with respect to their adjusted mean birth weights, adjusted mean weaning weights and estimated mean finishing weights derived from their respective adjusted mean carcass weights. As is apparent from FIG. 2, most of the differential growth between the long/long homozygous steers and the short/long heterozygous steers took place pre-weaning.

### EXAMPLE 3

### Additional Genetic Markers

Genetic markers in addition to the TG dinucleotide repeat described herein above were identified by the inventors as capable of being used in accordance herewith. One such example of a polymorphic site that can substitute for the TG-repeat-length-polymorphism is a G or A polymorphic site in exon 1A, shown on the bottom line of the alignment given in FIG. 3. This polymorphism may be efficiently detected by way of a restriction enzyme cut site polymorphism between the two alleles. The A allele contains a DraI restriction site that is not present in the G allele. This difference was used in a PCR/RFLP assay to distinguish the respective alleles, thereby yielding the same genotype information that was provided by the TG-repeat assay described above.

The two T or C upstream polymorphic sites identified in the first and second lines of FIG. 3 could similarly be used, as could a 0.35 kb retroposon located even further upstream. The retroposon is present in chromosomes with the longer TG repeat alleles, but is absent from chromosomes with only 11 consecutive TGs. Many other tightly linked polymorphic sites could also be used to obtain equivalent genotype information, as is described in detail herein above.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

Aggrey S E, Yao J, Sabour M P, Lin C Y, Zadworny D, Hayes J F, Kuhnlein U, 1999 “Markers within the regulatory region of the growth hormone receptor gene and their association with milk-related traits in Holsteins,” J. Hered. 90(1):148-51.

Bellus, J. Macromol. Sci. Pure Appl. Chem., RS3241(1):1355-1376, 1994.

Boggs, D. L. and R. A. Merkel. 1984. Live Animal Carcass Evaluation and Selection Manual (2nd Ed.) Kendall/Hunt Publishing Company, Dubuque, Iowa.

Boldman, K. G., L. A. Kriese, L. D. van Vleck, and S. D. Kachman. 1993. A manual for use of MTDFREML. ARS-USDA, Clay Center, NB.

Bradley, D. G., R. T. Loftus, P. Cunningham, and D. E. Machugh. 1998. Genetics and domestic cattle origins. Evolut. Anthropol. 6:79-86.

Edens, A. and F. Talamantes. 1998. Alternative processing of growth hormone receptor transcripts. Endocrine Rev. 19:559-582.

Frohman, In: PCR Protocols: A Guide To Methods And Applications, Academic Press, N.Y., 1990.

Gebhardt F., K. S. Zanker, and B. Brandt. 1999. Modulation of epidermal growth factor gene transcription by a polymorphic dinucleotide repeat in intron 1. J. Biol. Chem. 274:13176-13180.

Gingeras et al., 1990, “Unique features of the self-sustained sequence replication (3SR) reaction in the in vitro amplification of nucleic acids,” Ann Biol Clin (Paris) 48(7):498-501.

Heap, D., M. C. Lucy, R. J. Collier, C. K. Boyd, and W. C. Warren. 1995. Nucleotide sequence of the promoter and first exon of the somatotropin receptor gene in cattle. J. Anim. Sci. 73:1529.

Hillel et al., 1990, Genetics 124: 783-789.

Innis et al., “DNA sequencing with Thermus aquaticus DNA polymerase and direct sequencing of polymerase chain reaction-amplified DNA,” Proc Natl Acad Sci USA. 85(24):9436-9440, 1988.

Innis et al., “DNA sequencing with Thermus aquaticus DNA polymerase and direct sequencing of polymerase chain reaction-amplified DNA,” Proc Natl Acad Sci USA. 85(24):9436-9440, 1988.

Jiang, H., C. S. Okamura, and M. C. Lucy. 1999. Isolation and characterization of a novel promoter for the bovine growth hormone receptor gene. J. Biol. Chem. 274:7893-7900.

Kashi, Y, D. G. King, and M. Soller. 1997. Simple sequence repeats as a source of quantitative genetic variation. Trends Genet. 13:74-78.

Keightley and Hill, 1992, “Quantitative genetic variation in body size of mice from new mutations,” Genetics, 131:693-700.

King, D. G., M. Soller, and Y Kashi. 1997. Evolutionary tuning knobs. Endeavour 21: 36-40.

Kwoh et al., “Transcription-based amplification system and detection of amplified human immunodeficiency virus type 1 with a bead-based sandwich hybridization format, Proc Natl Acad Sci USA. 86(4):1173-1177, 1989.

Lagziel A., E. Lipkin, and M. Soller. 1996. Association between SSCP haplotype at the bovine growth hormone gene and milk protein percentage. Genetics 142:945-951.

Litt, M. and J. A. Luty, 1989. A hypervariable microsatellite revealed in vivo amplification of a dinucleotide repeat within the cardiac muscle actin gene. Am. J. Hum. Genet. 44:397-401.

Liu, J., C. K. Boyd, Y Kobayashi, C. C. Chase, A. C. Hammond, T. A. Olson, T. H. Elsasser, and M. C. Lucy. 1999. A novel phenotype for Laron dwarfism in miniature Bos indicus cattle suggests that the expression of growth hormone receptor 1A in liver is required for normal growth. Domest. Anim Endocrinol., accepted.

Lucy, M. C., G. S. Johnson, S. Shibuya, C. K. Boyd, and W. O. Herring. 1998. Polymorphic (GT)ₙ microsatellite in the bovine somatotrophin receptor gene promoter. J. Anim. Sci. 76:2209-2210.

Menon, R. K., D. A. Stephan, S. Manbir, S. M. Morris, and L. Zou. 1995. Cloning the promoter-regulatory region on the murine growth hormone receptor. J. Biol. Chem. 270:8851-8859.

O'Mahoney, V. O., M. R. Brandon, T. E., Adams. 1994. Identification of a liver-specific promoter for the ovine growth hormone receptor. Molecul. Cellul. Endocrinol. 101:129-139.

Ohara et al., “One-sided polymerase chain reaction: the amplification of cDNA,” Olson, M., L. Hood, C. Cantor, and D. Botstein, 1989. A common language for physical mapping of the human genome. Science 245:1434-1435.

Petlketsky, R. I., B. K. Chernov, and P. M. Rubtsov. Variants of the 5′-untranslated sequence of human growth hormone receptor MRNA. Molecul. Cellul. Endocrinol. 90:103-109.

Sambrook et al., In: Molecular Cloning: A Laboratory Manual, Vol. 1, Cold Spring Harbor Laboratory Press, Cold Spring Harbor, N.Y., Ch. 7,7.19-17.29, 1989.

Schwartzbauer, G., and R. K. Menon. 1998. Regulation of growth hormone receptor gene expression. Molec. Genet. Metabol. 63:243-253.

Shimajiri S., N. Nobuyuki, A. Tanimoto, Y. Murata, T. Hamada, K.-Y. Wang, Y.

Sasaguri. 1999. Shortened microsatellite d(CA)21 sequence down-regulates promoter activity of matrix metaloproteinase 9 gene. FEBS Let. 455:70-74.

Tautz, 1989, “Hypervariability of simple sequences as a general source for polymorphic DNA markers,” Nucleic Acids Res. 17:6563-6571.

Walker et al., “Strand displacement amplification—an isothermal, in vitro DNA amplification technique,” Nucleic Acids Res. 20(7): 1691-1696, 1992

Weber, J. L. and P. E. May. 1989. Abundant class of human DNA polymorphisms which can be typed using the polymerase chain reaction. Amer. J. Human Genet. 44:388-396.

Zhou, Y., B. C. Xu, H. G. Maheshwari, L. He, M. Reed, M. Lozykowski, S. Okada, L. Cataldo, K. Coschigamo. T. E. Wagner, G. Baumann, and J. J. Kopchick. 1997. A mammalian model for Laron syndrome produced by targeted disruption of the mouse growth hormone receptor/binding protein gene (the Laron mouse). Proc. Natl. Acad. Sci. USA. 94:13215-13220.

What is claimed is:

1. A method of obtaining a head of beef cattle comprising a genetic predisposition for increased or decreased carcass or weaning weight, the method comprising the steps of:
(a) assaying genetic material from at least a first head of beef cattle for a genetic polymorphism genetically linked to promoter P1 of exon 1A of the bovine growth hormone receptor gene, wherein said polymorphism is associated with increased or decreased carcass or weaning weight; and
(b) selecting a head of beef cattle comprising said polymorphism.

2. The method of claim 1, wherein said genetic polymorphism is further defined as genetically linked to exon 1A of the growth hormone receptor gene.

3. The method of claim 1, wherein said polymorphism is further defined as a polymorphism in a portion of the genome of said head of beef cattle corresponding to the nucleic acid sequence of SEQ ID NO: 3.

4. The method of claim 1, further defined as comprising assaying a plurality of beef cattle for said polymorphism.

5. The method of claim 1, wherein said polymorphism comprises a simple sequence length polymorphism.

6. The method of claim 5, wherein said simple sequence length polymorphism comprises a thymine-guanine dinucleotide repeat.

7. The method of claim 6, wherein said thymine-guanine dinucleotide repeat is further defined as flanked by the nucleic acid sequences of SEQ ID NO. 1 and SEQ ID NO. 2.

8. The method of claim 7, wherein said selecting comprises selecting a head of beef cattle comprising at least 12 copies of said thymine-guanine dinucleotide repeat.

9. The method of claim 7, wherein said selecting comprises selecting a head of beef cattle comprising between about 16 and about 20 copies of said thymine-guanine dinucleotide repeat.

10. The method of claim 7, wherein said selecting comprises selecting a head of beef cattle comprising less than 12 copies of said thymine-guanine dinucleotide repeat.

11. The method of claim 5, wherein said assaying is further defined as PCR.

12. The method of claim 5, wherein said assaying is further defined as comprising gel electrophoresis.

13. The method of claim 12, wherein said assaying comprises identifying specific amplification products by size.

14. The method of claim 1, wherein said head of beef cattle is further defined as a Bos indicus head of beef cattle.

15. The method of claim 1, wherein said head of beef cattle is further defined as a Bos taurus head of beef cattle.

16. The method of claim 1, wherein said polymorphism is further defined as a restriction fragment length polymorphism, simple sequence length polymorphism, amplified fragment length polymorphism, single nucleotide polymorphism or isozyme.

17. The method of claim 1, wherein said polymorphism is associated with increased carcass weight.

18. The method of claim 1, wherein said polymorphism is associated with decreased carcass weight.

19. The method of claim 1, wherein said polymorphism is associated with increased weaning weight.

20. The method of claim 1, wherein said polymorphism is associated with decreased weaning weight.

21. The method of claim 1, wherein said genetic material comprises genomic DNA.

22. The method of claim 1, wherein said genetic material is obtained from a developing fetus.

23. The method of claim 1, wherein said genetic material is obtained in vitro.

24. The method of claim 1, wherein said genetic material is obtained from an embryo.

25. The method of claim 24, wherein said selecting comprises embryo transfer of said embryo.

26. A method of breeding beef cattle to increase the probability of obtaining a progeny head of beef cattle comprising a predisposition for increased or decreased carcass or weaning weight, comprising the steps of:
(a) selecting a first parent head of beef cattle comprising a genetic polymorphism genetically linked to promoter P1 of exon 1A of the bovine growth hormone receptor gene, wherein said polymorphism is associated with increased or decreased carcass or weaning weight; and
(b) breeding said first parent head of beef cattle with a second parent head of beef cattle to obtain at least a first progeny head of beef cattle comprising said polymorphism associated with a genetic predisposition for increased carcass weight or weaning weight.

27. The method of claim 26, further comprising selecting said second parent head of beef cattle based on a genetic polymorphism genetically linked to promoter P1 of exon 1A of the bovine growth hormone receptor gene, wherein said polymorphism is associated with increased or decreased carcass or weaning weight.

28. The method of claim 26, wherein said genetic polymorphism is further defined as genetically linked to exon 1A of the growth hormone receptor gene.

29. The method of claim 26, wherein said polymorphism is further defined as a polymorphism in a portion of the genome of said head of beef cattle corresponding to the nucleic acid sequence of SEQ ID NO: 3.

30. The method of claim 26, wherein said polymorphism comprises a simple sequence length polymorphism.

31. The method of claim 30, wherein said simple sequence length polymorphism comprises a thymine-guanine dinucleotide repeat.

32. The method of claim 31, wherein said thymine-guanine dinucleotide repeat is further defined as flanked by the nucleic acid sequences of SEQ ID NO. 1 and SEQ ID NO. 2.

33. The method of claim 32, wherein said selecting comprises selecting a head of beef cattle comprising at least 12 copies of said thymine-guanine dinucleotide repeat.

34. The method of claim 32, wherein said selecting comprises selecting a head of beef cattle comprising between about 16 and about 20 copies of said thymine-guanine dinucleotide repeat.

35. The method of claim 32, wherein said selecting comprises selecting a head of beef cattle comprising less than 12 copies of said thymine-guanine dinucleotide repeat.

36. The method of claim 30, wherein said selecting comprises PCR.

37. The method of claim 30, wherein said selecting comprises gel electrophoresis.

38. The method of claim 37, wherein said selecting comprises identifying specific amplification products by size.

39. The method of claim 26, wherein one or both of said first parent head of beef cattle and said second parent head of beef cattle is further defined as a Bos indicus head of beef cattle.

40. The method of claim 26, wherein one or both of said first parent head of beef cattle and said second parent head of beef cattle is further defined as a Bos taurus head of beef cattle.

41. The method of claim 26, wherein said polymorphism is further defined as a restriction fragment length polymorphism, simple sequence length polymorphism, amplified fragment length polymorphism, single nucleotide polymorphism or isozyme.

42. The method of claim 26, wherein said polymorphism is associated with increased carcass weight.

43. The method of claim 26, wherein said polymorphism is associated with decreased carcass weight.

44. The method of claim 26, wherein said polymorphism is associated with increased weaning weight.

45. The method of claim 26, wherein said polymorphism is associated with decreased weaning weight.

46. The method of claim 26, wherein said first parent head of beef cattle is the sire and said second parent head of beef cattle is the dam.

47. The method of claim 26, wherein said first parent head of beef cattle is the dam and said second parent head of beef cattle is the sire.

48. The method of claim 26, further defined as comprising crossing said progeny head of beef cattle with a third head of beef cattle to produce a second generation progeny head of beef cattle.

49. The method of claim 26, further defined as comprising repeating step (a) and step (b) from about 2 to about 10 times.

50. The method of claim 49, wherein said first parent head of beef cattle is selected from a progeny head of beef cattle resulting from a previous repetition of said step (a) and said step (b) and wherein said second parent head of beef cattle is from a selected cattle breed into which one wishes to introduce said genetic predisposition for increased or decreased carcass or weaning weight.
